(19) United States
(12) Patent Application Publication
(10) Pub. No.: US 2018/0125856 A1
(43) Pub. Date: May 10, 2018

(54) USE OF GLUCOCORTICOID RECEPTOR ANTAGONIST AND SOMATOSTATIN ANALOGUES TO TREAT ACTH-SECRETING TUMORS

(71) Applicant: Corcept Therapeutics, Inc., Menlo Park, CA (US)

(72) Inventors: Andreas G. Moraitis, Menlo Park, CA (US); Joseph K. Belanoff, Menlo Park, CA (US)

(21) Appl. No.: 15/627,391

(22) Filed: Jun. 19, 2017

Related U.S. Application Data
(63) Continuation of application No. PCT/US2016/019646, filed on Feb. 25, 2016.
(60) Provisional application No. 62/127,153, filed on Mar. 2, 2015.

(51) Int. Cl. A61K 31/567; A61P 3/10; A61K 51/08; A61K 38/08; A61K 31/513; A61K 31/4745

(52) U.S. Cl. CPC A61K 31/567; A61P 3/10; A61K 31/4745; A61K 38/08; A61K 31/513; A61K 51/083

(57) ABSTRACT

Methods, compositions, and pharmaceutical formulations are provided for treatment of ACTH secreting tumors.

### CROSS-REFERENCES TO RELATED APPLICATIONS

This application is a U.S. National Phase Continuation Application of PCT/US2016/019646, filed Feb. 25, 2016, which claims priority to U.S. Provisional Application No. 62/127,153, filed Mar. 2, 2015, the contents of which are hereby incorporated in the entirety for all purposes.

### BACKGROUND OF THE INVENTION

Adrenocorticotropic hormone (ACTH) is a polypeptide-based hormone that is normally produced and secreted by the anterior pituitary gland. ACTH stimulates secretion of cortisol and other glucocorticoids by specialized cells of the adrenal cortex. In healthy mammals, ACTH secretion is tightly regulated. ACTH secretion can be positively regulated by corticotropin releasing hormone (CRH), which is released by the hypothalamus. ACTH secretion can be negatively regulated by cortisol and other glucocorticoids.

Aberrant ACTH-levels can lead to a wide variety of undesirable physiological conditions. For example, excess ACTH levels can cause excess secretion of cortisol, resulting in hypercortisolemia or Cushing's Syndrome. Excess ACTH can result from aberrant secretion of ACTH from tumors or other dysregulated cells.

ACTH-secreting tumors arising from pituitary corticotroph cells (Cushing's disease) exhibit poor prognosis, and cause hypercortisolemia. Similarly, non-pituitary or ectopic ACTH-secreting tumors can also cause Cushing's Syndrome. ACTH-secreting tumors can increase ACTH levels in a subject, resulting in excess cortisol secretion that can be associated with osteoporosis, infections, psychiatric disorders, muscle atrophy, fat accumulation, hypertension, hyperglycemia, or ultimately death.

ACTH-secreting pituitary tumors are generally treated by transsphenoidal pituitary tumor resection, pituitary-directed radiation, adrenalectomy and/or medical suppression of adrenal gland cortisol production. While transsphenoidal ACTH-secreting tumor resection yields 30-70% surgical cure rate, adenoma recurrence rate is high. Efficacies of other therapeutic modalities are limited by factors such as slow therapeutic response, development of pituitary insufficiency, and uncontrolled pituitary tumor growth in the face of adrenal gland resection or inhibition. Effective pharmacotherapy directly targeting corticotroph tumor growth and/or ACTH secretion remains a major challenge. Therefore, novel therapies are urgently needed for treating ACTH-secreting tumors.

### BRIEF SUMMARY OF THE INVENTION

In one aspect, the present invention provides a method of treating an adrenocorticotropic hormone (ACTH)-secreting tumor in a subject in need thereof, the method comprising simultaneously or sequentially administering to the subject: i) a glucocorticoid receptor antagonist (GRA); and ii) somatostatin or a somatostatin analog (SSA), in amounts effective to reduce secretion of ACTH by the tumor. In some embodiments, the patient suffers from Cushing's Disease. In some embodiments, the patient suffers from ectopic ACTH Syndrome. In some embodiments, the method comprises administering the GRA and SSA for at least two weeks (e.g., from two weeks to 1, 2, 3, 4, 5, 6, 7, 8, 9, 10, 11, 12, or more months). In some embodiments, the tumor is a neuroendocrine tumor. In some embodiments, the glucocorticoid receptor antagonist is a selective inhibitor of the glucocorticoid receptor.

In some embodiments, the glucocorticoid receptor antagonist comprises a steroidal backbone with at least one phenyl-containing moiety in the 11-β position of the steroidal backbone. In some cases, the phenyl-containing moiety in the 11-β position of the steroidal backbone is a dimethylaminophenyl moiety. In some cases, the glucocorticoid receptor antagonist is mifepristone. In some embodiments, the glucocorticoid receptor antagonist is selected from the group consisting of 11β-(4-dimethylaminoethoxyphenyl)-17α-propynyl-17β-hydroxy-4,9 estradien-3-one and (17α)-17-hydroxy-19-(4-methylphenyl)androsta-4,9(11)-dien-3-one. In some embodiments, the glucocorticoid receptor antagonist is (11β,17β)-11-(1,3-benzodioxol-5-yl)-17-hydroxy-17-(1-propynyl)estra-4,9-dien-3-one.

In some embodiments, the glucocorticoid receptor antagonist has a non-steroidal backbone. In some cases, the glucocorticoid receptor antagonist backbone is a cyclohexyl pyrimidine. In some cases, wherein the cyclohexyl pyrimidine has the following formula:

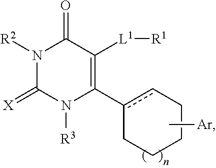

wherein the dashed line is absent or a bond; X is selected from the group consisting of O and S; R¹ is selected from the group consisting of cycloalkyl, heterocycloalkyl, aryl and heteroaryl, optionally substituted with from 1 to 3 R^{1a} groups; each R^{1a} is independently selected from the group consisting of H, C₁₋₆ alkyl, C₂₋₆ alkenyl, C₂₋₆ alkynyl, alkoxy, C₁₋₆ alkyl OR^{1b}, halogen, C₁₋₆ haloalkyl, haloaloxy, OR^{1b}, NR^{1b}R^{1c}, C(O)OR^{1b}, OC(O)R^{1b}, C(O)NR^{1b}R^{1c}, NR^{1b}C(O)R^{1c}, SO₂R^{1b}, SO₂NR^{1b}, cycloalkyl, heterocycloalkyl, aryl and heteroaryl; R^{1b} and R^{1c} are each independently selected from the group consisting of H and C₁₋₆ alkyl; R² is selected from the group consisting of H, C₁₋₆ alkyl, C₁₋₆ alkyl-OR^{1b}, C₁₋₆ alkyl NR^{1b}R^{1c} and C₁₋₆ alkylene heterocycloalkyl; R³ is selected from the group consisting of H and C₁₋₆ alkyl; Ar is aryl, optionally substituted with 1-4 R⁴ groups; each R⁴ is independently selected from the group consisting of H, C₁₋₆ alkyl, C₁₋₆ alkoxy, halogen, C₁₋₆ haloalkyl and C₁₋₆ haloalkoxy; L¹ is a bond or C₁₋₆ alkylene; and subscript n is an integer from 0 to 3, or salts and isomers thereof.

In some cases, the glucocorticoid receptor antagonist backbone is a fused azadeclin. In some cases, the fused azadeclin is a compound having the following formula:

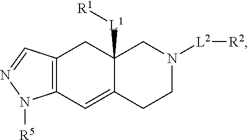

wherein L¹ and L² are members independently selected from a bond and unsubstituted alkylene; R¹ is a member selected from unsubstituted alkyl, unsubstituted heteroalkyl, unsubstituted heterocycloalkyl, —OR^{1A}, NR^{1C}R^{1D}, —C(O)NR^{1C}R^{1D}, and —C(O)OR^{1A}, wherein R^{1A} is a member selected from hydrogen, unsubstituted alkyl and unsubstituted heteroalkyl, R^{1C} and R^{1D} are members independently selected from unsubstituted alkyl and unsubstituted heteroalkyl, wherein R^{1C} and R^{1D} are optionally joined to form an unsubstituted ring with the nitrogen to which they are attached, wherein said ring optionally comprises an additional ring nitrogen; R² has the formula:

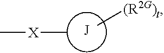

wherein R^{2G} is a member selected from hydrogen, halogen, unsubstituted alkyl, unsubstituted heteroalkyl, unsubstituted cycloalkyl, unsubstituted heterocycloalkyl, —CN, and —CF₃; J is phenyl; t is an integer from 0 to 5; X is —S(O₂)—; and R⁵ is phenyl optionally substituted with 1-5 R^{5A} groups, wherein R^{5A} is a member selected from hydrogen, halogen, —OR^{5A1}, S(O₂)NR^{5A2}R^{5A3}, —CN, and unsubstituted alkyl, wherein R^{5A1} is a member selected from hydrogen and unsubstituted alkyl, and R^{5A2} and R^{5A3} are members independently selected from hydrogen and unsubstituted alkyl, or salts and isomers thereof.

In some cases, the glucocorticoid receptor antagonist backbone is a heteroaryl ketone fused azadecalin or an octahydro fused azadecalin. In some cases, the heteroaryl ketone fused azadecalin has the formula:

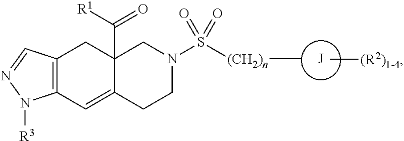

wherein R¹ is a heteroaryl ring having from 5 to 6 ring members and from 1 to 4 heteroatoms each independently selected from the group consisting of N, O and S, optionally substituted with 1-4 groups each independently selected from R^{1a}; each R^{1a} is independently selected from the group consisting of hydrogen, C₁₋₆ alkyl, halogen, C₁₋₆ haloalkyl, C₁₋₆ alkoxy, C₁₋₆ haloalkoxy, CN, N-oxide, C₃₋₈ cycloalkyl, and C₃₋₈ heterocycloalkyl; ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S; each R² is independently selected from the group consisting of hydrogen, C₁₋₆ alkyl, halogen, C_{1 6} haloalkyl, C_{1 6} alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl, wherein the heterocycloalkyl groups are optionally substituted with 1-4 R^{2c} groups; alternatively, two R² groups linked to the same carbon are combined to form an oxo group (═O); alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups; R^{2a} and R^{2b} are each independently selected from the group consisting of hydrogen and C₁₋₆ alkyl; each R^{2c} is independently selected from the group consisting of hydrogen, halogen, hydroxy, C₁₋₆ alkoxy, C₁₋₆ haloalkoxy, CN, and NR^{2a}R^{2b}; each R^{2d} is independently selected from the group consisting of hydrogen and C₁₋₆ alkyl, or two R^{2d} groups attached to the same ring atom are combined to form (═O); R³ is selected from the group consisting of phenyl and pyridyl, each optionally substituted with 1-4 R^{3a} groups; each R^{3a} is independently selected from the group consisting of hydrogen, halogen, and C₁₋₆ haloalkyl; and subscript n is an integer from 0 to 3; or salts and isomers thereof.

In some cases, the octahydro fused azadecalin has the formula:

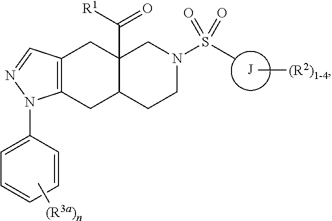

wherein R¹ is a heteroaryl ring having from 5 to 6 ring members and from 1 to 4 heteroatoms each independently selected from the group consisting of N, O and S, optionally substituted with 1-4 groups each independently selected from R^{1a}; each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl; ring J is selected from the group consisting of an aryl ring and a heteroaryl ring having from 5 to 6 ring members and from 1 to 4 heteroatoms each independently selected from the group consisting of N, O and S; each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl having from 1 to 3 heteroatoms each independently selected from the group consisting of N, O and S; alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups; R^{2a}, R^{2b} and R^{2c} are each independently selected from the group consisting of hydrogen and C₁₋₆ alkyl; each R^{3a} is independently halogen; and subscript n is an integer from 0 to 3, or salts and isomers thereof.

In some embodiments, the method comprises administering a somatostatin analog (SSA). In some cases, the somatostatin analog is selected from the group consisting of octreotide, octreotate, pasireotide, lanreotide, and derivatives thereof. In some cases, the somatostatin analog is radiolabeled. In some cases, the radiolabeled somatostatin analog is radiolabeled with a label suitable for imaging, such as, e.g., ¹¹¹In or ¹²³I. In some cases, the somatostatin analog is radiolabeled with a label suitable for radionuclide therapy, such as, e.g., ¹¹¹In, ¹³¹I, ⁹⁰Y, ¹⁷⁷Lu, or ²¹³Bi. In some cases, the therapeutic radionuclide is selected from the group consisting of ¹¹¹In, ⁹⁰Y, ¹⁷⁷Lu, and ²¹³Bi. In some cases, the therapeutic radionuclide is selected from the group consisting of ⁹⁰Y, ¹⁷⁷Lu, and ²¹³Bi. In some cases, the somatostatin analog is labeled with a radionuclide selected from the group consisting of ³²P, ⁴⁵Ti, ⁴⁸V, ⁴⁹V, ⁵⁹Fe, ⁶⁰CU, ⁶¹Cu, ⁶²Cu, ⁶⁴Cu, ⁶⁵Zn, ⁶⁷Cu, ⁶⁷Ga, ⁶⁸Ga, ⁷¹As, ⁷²As, ⁷⁶As, ⁷⁶Br, ⁷⁷As, ⁸⁹Sr, ⁹⁰Y, ^{99m}Tc, ¹¹¹In, ^{117m}Sn, ¹²³I, ¹²⁵I, ¹³¹I, ¹⁴⁹Pm, ¹⁵³Gd, ¹⁵³Pm, ¹⁵³Sm, ¹⁶⁶Ho, ¹⁷⁷Lu, ¹⁸⁶Re, ¹⁸⁸Re, ²⁰¹Tl, ²⁰³Pb, ²⁰⁹Pb, ²⁰⁹Bi, ²¹¹At, ²¹²Bi, ²¹²Pb, ²¹³Bi, ²²³Ra, and ²²⁵Ac. In some cases, the somatostatin analog is ¹²³I-Tyr³-octreotide, ¹¹¹In-DTPA-D-Phe¹-octreotide, [¹¹¹In-DTPA⁰]octreotide, [⁹⁰Y-DOTA, Tyr³]octreotide, or [¹⁷⁷Lu-DOTA, Tyr³]octreotate. In some cases, the subject in need thereof suffers from inoperable or metastatic ACTH-secreting tumor, or an inoperable and metastatic ACTH-secreting tumor. In some cases, the inoperable and/or metastatic ACTH-secreting tumor is a neuroendocrine tumor.

In some cases, the somatostatin analog is administered in a sustained release formulation. In some cases, the somatostatin analog is administered as octreotide LAR, lanreotide PR, or lanreotide autogel.

### BRIEF DESCRIPTION OF THE DRAWINGS

FIG. 1: illustrates the effect on adrenocorticotropic hormone (ACTH) and urinary free cortisol (UFC) levels of simultaneously or sequentially administering to a subject effective amounts of the glucocorticoid antagonist mifepristone (MIFE) and the somatostatin analog octreotide long-acting release (LAR). To convert values for UFC to nanomoles per 24 h, multiply by 2.76. To convert values for ACTH to picomoles per liter, multiply by 0.22.

### DETAILED DESCRIPTION OF THE INVENTION

### I. Introduction

The invention provides a novel treatment method for alleviating the effects of ACTH-secreting tumors by administering effective amounts of a glucocorticoid receptor antagonist (GRA) and a somatostatin receptor ligand such as somatostatin or a somatostatin analog (SSA).

### II. Definitions

The abbreviations used herein have their conventional meaning within the chemical and biological arts.

“Treat”, “treating” and “treatment” refer to any indicia of success in the treatment or amelioration of a pathology or condition, including any objective or subjective parameter such as abatement; remission; diminishing of symptoms or making the pathology or condition more tolerable to the patient; slowing in the rate of degeneration or decline; making the final point of degeneration less debilitating; or improving a patient's physical or mental well-being. The treatment or amelioration of symptoms can be based on objective or subjective parameters; including the results of a physical examination; histopathological examination (e.g., analysis of biopsied tissue); laboratory analysis of urine, saliva, an inferior petrosal sinus sample, serum, plasma, or blood (e.g., to detect cortisol or ACTH levels); or imaging (e.g., imaging of ACTH-secreting tumor or detectably labeled somatostatin analog). Effective treatment refers to a reduction in ACTH-secretion, a reduction in ACTH or cortisol levels, a reduction in ACTH-secreting tumor burden (e.g., ACTH-secreting tumor size, mass, volume, viability, or proliferation), or an increase in ACTH-secreting tumor cell death.

“Patient” or “subject in need thereof” refers to a person having, or suspected of having, a neuroendocrine tumor, an ACTH-secreting tumor, or an ACTH-secreting neuroendocrine tumor. An ACTH-secreting tumor can be identified and/or monitored by detection of the tumor, or detection of symptoms caused by an ACTH-secreting tumor. A neuroendocrine tumor can be detected and/or monitored by detection of the tumor or detection of symptoms caused by the tumor.

As used herein, the term “ACTH-secreting tumor” refers to an adenoma, adenocarcinoma, neuroendocrine, pituitary, or other tumor that secretes ACTH. In some cases, the ACTH-secreting tumor can cause an increase in blood, plasma, or serum levels of ACTH or blood, plasma, serum, or urinary (e.g., urinary free) cortisol levels in a subject having the ACTH-secreting tumor as compared to a subject that does not have an ACTH-secreting tumor. In some cases, the ACTH-secreting tumor does not respond to suppression or negative regulation of ACTH secretion by cortisol or other glucocorticoid receptor agonists (e.g., dexamethasone).

As used herein, the term “simultaneously or sequentially administering” refers to administration of a GRA compound and somatostatin receptor ligand compound (e.g., somatostatin or somatostatin analog (SSA)) such that the two compounds are in the body at the same time in amounts effective to treat an ACTH-secreting tumor.

As used herein, the term “effective amount,” “amounts effective,” or “therapeutically effective amount” refers to an amount or amounts of one or more pharmacological agents effective to treat, eliminate, or mitigate at least one symptom of the disease being treated. In some cases, “effective amount,” “amounts effective,” or “therapeutically effective amount” can refer to an amount of a functional agent or of a pharmaceutical composition useful for exhibiting a detectable therapeutic or inhibitory effect. The effect can be detected by any assay method known in the art. In some cases, the amounts effective, or the like, refer to amounts effective to reduce ACTH levels. In some cases, the amounts effective, or the like, refer to amounts effective to reduce cortisol (e.g., serum cortisol, salivary cortisol, or urinary free cortisol) levels. In some cases, the amounts effective, or the like, refer to amounts effective to reduce ACTH levels or cortisol levels, or a combination thereof, by at least 10%, 20%, 30%, 40%, 50%, 60%, 75%, 90%, 99%, or more.

As used herein, the term “effective to reduce secretion of ACTH by the tumor” refers to a method, treatment, composition, or amount that can reduce secretion of ACTH by pituitary, neuroendocrine, or other tumor as compared to the secretion of ACTH by such a tumor in the absence of the method, treatment, composition, or amount.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non limiting examples of pharmaceutically acceptable excipients include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical excipients are useful in the present invention.

“Glucocorticoid receptor” (“GR”) refers to the type II GR which specifically binds to cortisol and/or cortisol analogs such as dexamethasone (See, e.g., Turner & Muller, J Mol Endocrinol Oct. 1, 2005 35 283-292). The GR is also referred to as the cortisol receptor. The term includes isoforms of GR, recombinant GR and mutated GR. Inhibition constants (Kᵢ) against the human GR receptor type II (Genbank: P04150) are between 0.0001 nM to 1,000 nM; preferably between 0.0005 nM to 10 nM, and most preferably between 0.001 nM to 1 nM.

“Glucocorticoid receptor antagonist” refers to any composition or compound which partially or completely inhibits (antagonizes) the binding of a glucocorticoid receptor (GR) agonist, such as cortisol, or cortisol analogs, synthetic or natural, to a GR. A “specific glucocorticoid receptor antagonist” refers to any composition or compound which inhibits any biological response associated with the binding of a GR to an agonist. By “specific,” the drug preferentially binds to the GR rather than other nuclear receptors, such as mineralocorticoid receptor (MR), androgen receptor (AR), or progesterone receptor (PR). It is preferred that the specific glucocorticoid receptor antagonist bind GR with an affinity that is 10× greater ( 1/10^{th} the K_{d} value) than its affinity to the MR, AR, or PR, both the MR and PR, both the MR and AR, both the AR and PR, or to the MR, AR, and PR. In a more preferred embodiment, the specific glucocorticoid receptor antagonist binds GR with an affinity that is 100× greater ( 1/100^{th} the K_{d} value) than its affinity to the MR, AR, or PR, both the MR and PR, both the MR and AR, both the AR and PR, or to the MR, AR, and PR.

As used herein, the phrase “steroidal backbone” in the context of glucocorticoid receptor antagonists containing such refers to glucocorticoid receptor antagonists that contain modifications of the basic structure of cortisol, an endogenous steroidal glucocorticoid receptor ligand. The basic structure of a steroidal backbone is provided as Formula I:

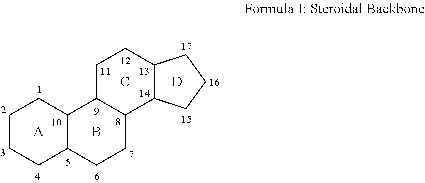

The two most commonly known classes of structural modifications of the cortisol steroid backbone to create glucocorticoid antagonists include modifications of the 11-β hydroxy group and modification of the 17-β side chain (See, e. g., Lefebvre (1989) J. Steroid Biochem. 33: 557-563).

As used herein, the phrase “non-steroidal backbone” in the context of glucocorticoid receptor antagonists containing such refers to glucocorticoid receptor antagonists that do not share structural homology to, or are not modifications of, cortisol. Such compounds include synthetic mimetics and analogs of proteins, including partially peptidic, pseudopeptidic and non-peptidic molecular entities.

Non-steroidal GRA compounds also include glucocorticoid receptor antagonists having a cyclohexyl-pyrimidine backbone, a fused azadecalin backbone, a heteroaryl ketone fused azadecalin backbone, or an octahydro fused azadecalin backbone. Exemplary glucocorticoid receptor antagonists having a cyclohexyl-pyrimidine backbone include those described in U.S. Pat. No. 8,685,973. Exemplary glucocorticoid receptor antagonists having a fused azadecalin backbone include those described in U.S. Pat. Nos. 7,928,237; and 8,461,172. Exemplary glucocorticoid receptor antagonists having a heteroaryl ketone fused azadecalin backbone include those described in U.S. Pat. No. 8,859,774. Exemplary glucocorticoid receptor antagonists having an octohydro fused azadecalin backbone include those described in U.S. Provisional Patent Appl. No. 61/908,333, entitled Octahydro Fused Azadecalin Glucocorticoid Receptor Modulators, Attorney Docket No. 85178-887884 (007800US), filed on Nov. 25, 2013; and U.S. Patent Application Publication No. 2015/0148341.

As used herein, the term somatostatin receptor refers to a class of G-protein coupled seven transmembrane receptors that bind somatostatin. There are five somatostatin receptor sub-types, referred to as SSTR1-SSTR5 respectively. See, e.g., Trends Pharmacol Sci. 1995 March; 16(3):86-8.

As used herein, the terms “somatostatin receptor ligand,” or “somatostatin or somatostatin analog” refer to any ligand of any one of the somatostatin receptor subtypes (SSTR1-SSTR5). In some cases, the ligand is somatostatin. Somatostatin is an inhibitory polypeptide with two primary biologically active forms SST14 and SST28. In some cases, the ligand is a pre- or pre-pro form of somatostatin, or an analog thereof. In some cases, the somatostatin ligand is a somatostatin analog. Somatostatin analogs can be agonists or antagonists of one or more somatostatin receptors. In some cases, the somatostatin ligand preferentially binds or activates somatostatin receptor type 2 (SSTR2). In some cases, the somatostatin receptor ligand preferentially binds or activates somatostatin receptor type 5 (SSTR5). In some cases, the somatostatin receptor ligand preferentially binds or activates SSTR2 and SSTR5. In some cases, the somatostatin receptor ligand preferentially binds or activates SSTR2, SSTR3, and SSTR5. The somatostatin receptor ligand can be provided or administered in a long acting, prolonged, or slow release formulation.

Where substituent groups are specified by their conventional chemical formulae, written from left to right, they equally encompass the chemically identical substituents that would result from writing the structure from right to left, e.g., —CH₂O— is equivalent to —OCH₂—.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec.butyl, tert.butyl, pentyl, isopentyl, hexyl, etc.

“Alkoxy” refers to an alkyl group having an oxygen atom that connects the alkyl group to the point of attachment: alkyl-O—. As for the alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Halogen” refers to fluorine, chlorine, bromine and iodine.

“Haloalkyl” refers to alkyl, as defined above, where some or all of the hydrogen atoms are replaced with halogen atoms. As for the alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethane includes 1,1,1-trifluoromethyl.

“Haloalkoxy” refers to an alkoxy group where some or all of the hydrogen atoms are substituted with halogen atoms. As for the alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl.

“Heterocycloalkyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxalidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxazolidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, N-oxide, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring heteroatoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Heteroatoms” refers to O, S or N.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Isomers” refers to compounds with the same chemical formula but which are structurally distinguishable.

“Tautomer” refers to one of two or more structural isomers which exist in equilibrium and which are readily converted from one form to another.

As used herein, the terms “sustained release,” “slow release,” “long acting,” “prolonged release,” and the like refer to a formulation containing at least one active ingredient (e.g., somatostatin analog, GRA, or combination thereof) formulated to maintain a therapeutic concentration of active ingredient(s) in a patient for a longer period of time in comparison to formulations that are not designed for such sustained release. In some cases, the sustained release formulation maintains therapeutic concentration of one or more active ingredient(s) for, or for at least, one week, two weeks, three weeks, four weeks, five weeks, or six weeks. In some cases, the sustained release formulation is administered to a patient every one, two, three, four, five, or six weeks. Exemplary sustained release formulations include, but are not limited to octreotide LAR, prolonged release lanreotide, and lanreotide autogel.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions.

### III. Methods

The present invention provides a method of treating an adrenocorticotropic hormone (ACTH)-secreting tumor in a subject in need thereof. In one aspect, the method comprises administering to the subject a glucocorticoid receptor antagonist GRA and somatostatin, a somatostatin analog (SSA), or a somatostatin receptor ligand, in amounts effective to reduce secretion of ACTH by the tumor. The administering can be simultaneous administration in which the GRA and the somatostatin, SSA or somatostatin receptor ligand are administered in a formulation containing both compounds. Alternatively, the GRA can be administered and then the somatostatin, SSA, or somatostatin receptor ligand can be administered. As yet another alternative, the somatostatin, SSA, or somatostatin receptor ligand can be administered and then the GRA administered.

### A. ACTH-Secreting Tumors

### 1) Types of ACTH-Secreting Tumors

Methods and compositions described herein are useful for treating a wide variety of ACTH-secreting tumors. ACTH-secreting tumors include, but are not limited to, tumors that secrete ACTH and express one or more somatostatin receptors (e.g., one of SSTR1-5, or a combination thereof). In some cases, expression of one or more somatostatin receptor subtypes (e.g., expression of one of SSTR1-5, or a combination thereof) is upregulated in an ACTH-secreting tumor after administration of a glucocorticoid receptor antagonist. In some cases, somatostatin receptor expression (e.g., expression of one of SSTR1-5, or a combination thereof) is undetectable prior to administration of a glucocorticoid receptor antagonist and detectable after administration of a glucocorticoid receptor antagonist. In some cases, The types of ACTH-secreting tumors that can be treated by the methods and compositions described herein include, but are not limited to, adenomas, adenocarcinomas, pituitary adenomas or pituitary adenocarcinomas, carcinoid tumors, neuroendocrine tumors, or combinations thereof.

The ACTH-secreting tumor can be a benign or malignant neuroendocrine tumor (NET) arising from neuroendocrine cells which are found throughout the body in organs such as the pituitary, thyroid, adrenals, pancreas, the lungs and the gastrointestinal tract. In some cases, the ACTH-secreting tumor is an adenoma or adenocarcinoma. An adenoma is a benign tumor arising from epithelial tissue of glandular origin, having glandular characteristics, or both. Adenomas can arise in a variety of glandular tissues, such as adrenal glands and pituitary glands. Some adenomas arise from non-glandular tissues, but express glandular tissue structure. Although adenomas are benign, over time they may transform to become malignant, at which point they are called adenocarcinomas. However, even while benign, adenomas have the potential to cause serious health complications by compressing other structures (e.g., compressing brain or ocular nerve structures) or by producing large amounts of hormones (e.g., ACTH) in an unregulated, non-feedback-dependent manner. Even very small adenomas have the potential to secrete sufficient amounts of one or more hormones to cause clinical symptoms.

The ACTH-secreting tumor can be a pituitary adenoma or pituitary adenocarcinoma. ACTH-secreting pituitary tumors can cause Cushing's Disease. As such, methods and compositions described herein can treat or relieve one or more symptoms of Cushing's Disease.

In some cases, the ACTH-secreting pituitary adenoma or adenocarcinoma has aberrant somatostatin receptor expression as compared to normal pituitary cells. Normal adult pituitary cells express somatostatin receptors SSTR1, 2, 3, and 5. In normal cells, SSTR5 is the most highly expressed somatostatin receptor sub-type, and SSTR4 is expressed at very low levels. Approximately 85% of ACTH-secreting pituitary adenomas express SSTR2 and SSTR5, while approximately 63% express SSTR1. See, Cuevas-Ramos & Fleseriu J. Mol. Endocrinol. (2014) 52, R223-R240.

In ACTH-secreting pituitary adenomas or adenocarcinomas, SSTR5 can remain the most abundant somatostatin receptor; however, somatostatin receptor expression (e.g., expression of any one of SSTR1-5, or a combination thereof) can be reduced. For example, expression of any one of SSTR1-5, or a combination thereof can be reduced due to hypercortisolism-induced down-regulation. In some cases, SSTR2 expression is reduced due to hypercortisolism induced downregulation. In some cases, the ACTH-secreting pituitary tumors increase somatostatin receptor expression (e.g., expression of any one of SSTR1-5, or a combination thereof) in response to GRA administration by blocking or mitigating hypercortisolism-induced down-regulation of SSTR expression. In some cases, SSTR2 expression is increased by GRA administration by blocking or mitigating hypercortisolism-induced down-regulation of SSTR2 expression.

The ACTH-secreting tumor can be a neuroendocrine tumor or a carcinoid tumor. Neuroendocrine tumors arise from cells of the endocrine or nervous systems. Neuroendocrine tumors can occur in any area or region of the body. However, neuroendocrine tumors are most often found in the intestine, pancreas, or lung. Neuroendocrine tumors can be classified as well-differentiated benign, well-differentiated uncertain, well-differentiated low-grade malignant, or poorly differentiated malignant tumors. The ACTH-secreting neuroendocrine tumor or carcinoid tumor can cause ectopic Cushing's Syndrome. As such, methods and compositions described herein can treat or relieve one or more symptoms of ectopic Cushing's Syndrome.

Neuroendocrine tumors can include neuroendocrine tumors of the anterior pituitary; neuroendocrine thyroid tumors, such as medullary carcinomas; parathyroid tumors; thymus and mediastinal carcinoid tumors; pulmonary neuroendocrine tumors (e.g., bronchial tumors, pulmonary carcinoid tumors such as typical carcinoid, or atypical carcinoid tumors, small-cell lung cancer, and large cell neuroendocrine carcinomas of the lung); extrapulmonary small cell carcinomas; gastroenteropancreatic neuroendocrine tumors (e.g., foregut, midgut, or hindgut gastroenteropancreatic neuroendocrine tumors); neuroendocrine tumors of the liver or gallbladder; adrenal tumors; addrenomedullary tumors; pheochromocytomas; peripheral nervous system tumors (e.g., schwannoma, paraganglioma; or neuroblastoma); tumors of the breast; genitourinary tract tumors (e.g., urinary tract carcinoid tumors, ovarian tumors, neuroendocrine tumors of the cervix, or testicular tumors); Merkel cell carcinoma of the skin; multiple endocrine neoplasia type 1 or 2 tumors; tumors resulting from von Hippel-Lindau disease; neurofibromatosis type 1 tumors; tumors associated with tuberous sclerosis; tumors associated with or caused by Carney complex; or combinations thereof.

In some cases, the neuroendocrine or carcinoid tumor, such as one of the neuroendocrine or carcinoid tumors described herein, expresses one or more somatostatin receptor subtypes (e.g., one of SSTR1-5, or a combination thereof). In some cases, the neuroendocrine or carcinoid tumor exhibits downregulated expression of one or more somatostatin receptor subtypes (e.g., one of SSTR1-5, or a combination thereof). In some cases, the neuroendocrine or carcinoid tumor exhibits downregulated expression of one or more somatostatin receptor subtypes (e.g., one of SSTR1-5, or a combination thereof) in response to hypercortisolism. In some cases, the neuroendocrine or carcinoid tumor exhibits downregulated expression of SSTR2 in response to hypercortisolism. In some cases, administration of a GRA can increase expression of one or more somatostatin receptor subtypes (e.g., one of SSTR1-5, or a combination thereof) by blocking or mitigating hypercortisolism-induced down-regulation of SSTR expression. In some cases, SSTR2 expression is increased by GRA administration by blocking or mitigating hypercortisolism-induced down-regulation of SSTR2 expression.

### 2) Detection or Diagnosis of ACTH-Secreting Tumors

ACTH-secreting tumors can be detected by a variety of means. For example, ACTH-secreting tumors generally result in the presence of excess ACTH, resulting in hypercortisolism. Thus, ACTH-secreting tumors can be identified based on the presence of symptoms of hypercortisolism (e.g., symptoms of Cushing's Disease or ectopic Cushing's Syndrome). Such symptoms include, but are not limited to one or more of the following: weight gain, high blood pressure, poor short term memory, poor concentration, irritability, excess hair growth, impaired immunological function, ruddy complexion, extra fat in the neck region, moon face, fatigue, red stretch marks, irregular menstruation, or a combination thereof. Symptoms of hypercortisolism can additionally or alternatively include without limitation one or more of the following: insomnia, recurrent infection, thin skin, easy bruising, weak bones, acne, balding, depression, hip or shoulder weakness, swelling of the extremities, diabetes mellitus, elevated white blood cell count, hypokalemic metabolic alkalosis, or a combination thereof.

In some cases, symptoms of hypercortisolism (e.g., Cushing's Disease or Cushing's Syndrome) are difficult to differentiate from other causes. Therefore, biochemical tests can be useful to determine the presence or absence of hypercortisolism, indicating the presence or absence of ACTH-secreting tumors respectively. Alternatively, biochemical tests can be used to directly detect the presence of excess ACTH. As such biochemical tests useful for the detection of ACTH-secreting tumors include, but are not limited to, tests that measure ACTH, cortisol, or a combination thereof. In some cases, salivary or blood serum cortisol levels are measured. In some cases, urinary free cortisol (UFC), e.g., 24-hour UFC is measured.

In some cases, ACTH is measured by bilateral inferior petrosal sinus sampling (BIPSS). In some cases, ACTH is measured by bilateral internal jugular vein sampling (BIJVS). In some cases, ACTH levels from BIPSS and/or BIJVS are compared to a peripherally obtained sample. The inferior petrosal sinus is where the pituitary gland drains. Therefore, a sample from this area that has high ACTH levels can suggest the presence of an ACTH-secreting tumor in the pituitary gland (i.e., Cushing's Disease). A low level of ACTH measured from the inferior pertrosal sinus can indicate the presence of an ACTH-secreting tumor that does not reside in the pituitary (e.g., ectopic Cushing's Syndrome). In some cases, detection of a central to periphery ACTH gradient can indicate whether the ACTH-secreting tumor is pituitary or non-pituitary.

In some cases, BIPSS and/or BIJVS is performed after administration of corticotropin-releasing hormone (CRH), desmopressin (DDAVP), or a combination thereof. These agents can increase ACTH secretion in active ACTH-producing pituitary tumors. In some cases, sampling is performed before and after administration of CRH, DDAVP, or a combination thereof. In some cases, a central to periphery ACTH gradient of more than 2 before and more than 3 after the administration of CRH or DDAVP indicates the presence of an ACTH secreting pituitary tumor. In some cases, gradients less than 2 before or less than 3 after the administration of CRH or DDAVP indicate a non-pituitary ACTH-secreting tumor.

ACTH-secreting tumors of pituitary or non-pituitary origin or location can be differentiated from other diseases or conditions that cause hypercortisolism or Cushing's-like symptoms by their ACTH-dependence. ACTH-dependent Cushing's Syndrome can indicate ectopic or pituitary Cushing's. ACTH-independent Cushing's Syndrome can indicate adrenal dysfunction (e.g., the presence of an adrenal adenoma or carcinoma). ACTH-independent Cushing's can be indicated by a low or undetectable plasma ACTH level in combination with a simultaneously elevated serum cortisol level. For example, a plasma ACTH level of less than 5 pg/mL (e.g., measured by an immunoradiometric assay) in a subject with high cortisol levels is suggestive of a primary adrenal tumor. In contrast, an ACTH level greater than 10-20 pg/mL is consistent with ACTH-dependent Cushing syndrome.

An overnight dexamethasone suppression test and/or high-dose dexamethasone test may be useful to diagnose the source of hypercortisolism, e.g., when baseline ACTH levels are indeterminate. These tests also help in determining whether a patient who has ACTH-dependent disease has pituitary-dependent or ectopic ACTH disease. For example, in the 8 mg overnight dexamethasone suppression test, individuals can ingest 8 mg dexamethasone orally in the evening of the first day, with measurement of cortisol levels early the next day. A baseline morning cortisol measurement can also obtained the morning prior to ingesting dexamethasone. Suppression of serum cortisol levels to less than 50% of baseline is suggestive of a pituitary source of ACTH rather than ectopic ACTH or primary adrenal disease.

As another example, in a 48-hour high-dose dexamethasone suppression test, patients ingest 2 mg dexamethasone every 6 hours for 8 doses. A decrease in urinary free cortisol of greater than 50% is suggestive of an anterior pituitary adenoma rather than ectopic ACTH or a primary adrenal tumor. The more stringent criterion of a 90% decrease in urinary free cortisol levels excludes the diagnosis of ectopic ACTH and has almost 100% specificity for anterior pituitary disease.

Testing with corticotropin releasing hormone (CRH) can be used in the differential diagnosis of ACTH-dependent Cushing syndrome. In most subjects with pituitary ACTH secretion, the intravenous administration of CRH causes a rise in plasma ACTH and cortisol levels. In subjects with ectopic secretion of ACTH, CRH generally does not affect ACTH or cortisol levels. In a CRH-test, ACTH and cortisol samples are obtained before administration of ovine CRH (oCRH), and subsequently at 15, 30, 45, 60, 90, and 120 minutes after administration of 1 mcg/kg of CRH. A rise of more than 20% in peak plasma cortisol level or a rise of more than 50% in peak ACTH level after oCRH is consistent with pituitary ACTH-dependent Cushing syndrome. Sensitivity and specificity are 91% and 95%, respectively, for cortisol measurements and 86% and 95% for ACTH measurements, respectively.

ACTH-secreting tumors can also be detected via imaging studies, such as computed tomography (CT) scanning, magnetic resonance imaging, scintigraphy, single photon emission computerized tomography (SPECT), ultrasound imaging, or a combination thereof. Generally, imaging studies are performed after biochemical tests have been performed and specific types of ACTH-secreting tumors are suspected or indicated. For example, if biochemical testing suggests ectopic ACTH-secreting tumors, then imaging can be used to scan for these tumors, e.g., in the chest or abdominal area where ectopic ACTH-secreting tumors most often arise. Similarly, if biochemical testing suggests pituitary ACTH secreting tumors, then imaging can be restricted to the pituitary gland and surrounding tissues.

For example, if a pituitary source of excess ACTH is suspected, subjects can undergo a contrast-enhanced magnetic resonance imaging (MM) study of the pituitary. Unfortunately, normal-appearing pituitaries may occur in some patients with Cushing disease due to either diffuse hyperplasia of ACTH-producing cells or small microadenomas that do not appear on imaging studies. In the latter case, ACTH lateralization during a bilateral inferior petrosal sinus sampling (BIPSS) or bilateral internal jugular vein sampling (BIJVS) study may be useful in lateralizing an occult lesion and in guiding surgical therapy.

As another example detectably labeled somatostatin or somatostatin analogues (SSAs) can be used to identify ACTH-secreting tumors (e.g., ectopic ACTH-secreting tumors such as neuroendocrine tumors). Such somatostatin or SSAs can include labeled octreotide. For example, ¹²³I-Tyr³, ¹¹¹In-DTPA-D-Phe¹-octreotide, or [¹¹¹In-DTPA⁰]octreotide can be administered to a patient, allowed to bind to somatostatin receptor expressing ACTH-secreting tumors, and detected. Further examples of somatostatins and somatostatin analogues, including radionuclide labeled somatostatin analogues useful for imaging and/or radionuclide therapy include those disclosed in Baldelli et al. Front Endocrinol (Lausanne). 2014 Feb. 7; 5:7. In some cases, identification of ACTH-secreting tumors with a detectably labeled somatostatin or somatostatin analogue is augmented by administration of a glucocorticoid receptor antagonist (GRA) (e.g., mifepristone). In some cases, administration of a GRA can de-repress expression of one or more somatostatin receptors by the ACTH-secreting tumor cells (de Bruin et al, J Clin Endocrinol Metab, February 2012, 97(2):455-46).

### 3) Determining Treatment Efficacy

Any one or more of the foregoing detection or diagnostic methods described herein, or known generally in the art, can be used to assess treatment effect. In some embodiments, an ACTH-secreting tumor is treated in a subject in need thereof by administering a GRA and a somatostatin or somatostatin analog (SSA) in amounts effective to treat the ACTH-secreting tumor, and the treatment is monitored to determine efficacy. For example, efficacy can be indicated by detecting a decrease in ACTH levels, a decrease in hypercortisolism (e.g., a decrease in serum, urinary free, or salivary cortisol levels, or a decrease in symptoms of high cortisol levels), or a reduction in tumor burden. In some cases, the reduction in tumor burden is indicated by a reduction in size, mass, or volume of a tumor, or a reduction in symptoms caused by the tumor mass. For example, where a tumor mass physically impinges upon the optic nerve, effective treatment can be indicated by a reduction in visual field defects. In some cases, the reduction in tumor burden is indicated by a reduction in ACTH-secreting tumor cell proliferation or viability, or by an increase in ACTH-secreting tumor cell death.

### B. Glucocorticoid Receptor Antagonists

The methods of the present invention generally provide administering a glucocorticoid receptor antagonist. In some cases, the glucocorticoid receptor antagonist is a specific glucocorticoid receptor antagonist. As used herein, a specific glucocorticoid receptor antagonist refers to a composition or compound which inhibits any biological response associated with the binding of a glucocorticoid receptor to an agonist by preferentially binding to the glucocorticoid receptor rather than another nuclear receptor (NR). In some embodiments, the specific glucocorticoid receptor antagonist binds preferentially to glucocorticoid receptor rather than the mineralocorticoid receptor (MR), androgen receptor (AR), or progesterone receptor (PR). In an exemplary embodiment, the specific glucocorticoid receptor antagonist binds preferentially to glucocorticoid receptor rather than the mineralocorticoid receptor (MR). In another exemplary embodiment, the specific glucocorticoid receptor antagonist binds preferentially to glucocorticoid receptor rather than the progesterone receptor (PR). In another exemplary embodiment, the specific glucocorticoid receptor antagonist binds preferentially to glucocorticoid receptor rather than the androgen receptor (AR). In yet another exemplary embodiment, the specific glucocorticoid receptor antagonist binds preferentially to glucocorticoid receptor in comparison to MR and PR, MR and AR, or MR, PR, and AR.

In a related embodiment, the specific glucocorticoid receptor antagonist binds to the glucocorticoid receptor with a dissociation constant (K_{d}) that is or is less than 1/10^{th} the K_{d} (i.e., at least 10× greater affinity than) for another nuclear receptor (e.g., AR; MR; PR; MR and PR; MR and AR; or MR; PR; and AR). In another embodiment, the specific glucocorticoid receptor antagonist binds to the glucocorticoid receptor with a dissociation constant (K_{d}) that is or is less than 1/100^{th} the K_{d} (i.e., at least 100× greater affinity than) for the other nuclear receptor (e.g., AR; MR; PR; MR and PR; MR and AR; or MR; PR; and AR). In another embodiment, the specific glucocorticoid receptor antagonist binds to the glucocorticoid receptor with a dissociation constant (K_{d}) that is or is less than 1/1000^{th} the K_{d} (i.e., at least 1000× greater affinity than) for the other nuclear receptor (e.g., AR; MR; PR; MR and PR; MR and AR; or MR; PR; and AR).

### 1) Exemplary Glucocorticoid Receptor Antagonists

Generally, treatment can be provided by administering an effective amount of a glucocorticoid receptor antagonist (GRA) of any chemical structure or mechanism of action and a somatostatin, SSA, or somatostatin receptor ligand of any chemical structure or mechanism of action. Provided herein, are classes of exemplary GRAs and specific members of such classes. However, one of skill in the art will readily recognize other related or unrelated GRAs that can be employed in the treatment methods described herein.

### a) GRAs Having a Steroidal Backbone

In some embodiments, an effective amount of a GRA with a steroidal backbone is administered to a subject for treatment of an ACTH-secreting tumor. Steroidal GRAs can be obtained by modification of the basic structure of glucocorticoid agonists, i.e., varied forms of the steroid backbone. The structure of cortisol can be modified in a variety of ways. The two most commonly known classes of structural modifications of the cortisol steroid backbone to create GRAs include modifications of the 11-β hydroxy group and modification of the 17-β side chain (See, e.g., Lefebvre, J. Steroid Biochem. 33:557-563, 1989).

Examples of steroidal GR antagonists include androgen-type steroidal compounds as described in U.S. Pat. No. 5,929,058, and the compounds disclosed in U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; 5,616,458, 5,696,127, and 6,303,591. Such steroidal GR antagonists include cortexolone, dexamethasone-oxetanone, 19-nordeoxycorticosterone, 19-norprogesterone, cortisol-21-mesylate; dexamethasone-21-mesylate, 11β-(4-dimethylaminoethoxyphenyl)-17α-propynyl-17β-hydroxy-4,9-estradien-3-one (RU009), and (17α)-17-hydroxy-19-(4-methylphenyl)androsta-4,9(11)-dien-3-one (RU044).

Other examples of steroidal antiglucocorticoids are disclosed in Van Kampen et al. (2002) Eur. J. Pharmacol. 457(2-3):207, WO 03/043640, EP 0 683 172 B1, and EP 0 763 541 B1, each of which is incorporated herein by reference. EP 0 763 541 B1 and Hoyberg et al., Int'l J. of Neuro-psychopharmacology, 5:Supp. 1, 5148 (2002); disclose the compound (11β,17β)-11-(1,3-benzodioxol-5-yl)-17-hydroxy-17-(1-propynyl)estra-4,9-dien-3-one (ORG 34517) which in one embodiment, is administered in an amount effective to treat an ACTH-secreting tumor in a subject.

### i. Removal or Substitution of the 11-β Hydroxy Group

Glucocorticoid antagonists with modified steroidal backbones comprising removal or substitution of the 11-β hydroxy group are administered in one embodiment of the invention. This class includes natural GRAs, including cortexolone, progesterone and testosterone derivatives, and synthetic compositions, such as mifepristone (Lefebvre, et al. supra). Preferred embodiments of the invention include all 11-β aryl steroid backbone derivatives because, in some cases, these compounds can be devoid of progesterone receptor (PR) binding activity (Agarwal, FEBS 217:221-226, 1987). In another embodiment an 11-β phenyl-aminodimethyl steroid backbone derivative, which is both an effective anti-glucocorticoid and anti-progesterone agent, is administered. These compositions can act as reversibly-binding steroid receptor antagonists. For example, when bound to a 11-β phenyl-aminodimethyl steroid, the steroid receptor can be maintained in a conformation that cannot bind its natural ligand, such as cortisol in the case of GR (Cadepond, 1997, supra).

Synthetic 11-beta phenyl-aminodimethyl steroids include mifepristone, also known as RU486, or 17-β-hydrox-11-β-(4-dimethyl-aminophenyl)17-α-(1-propynyl)estra-4,9-dien-3-one). Mifepristone has been shown to be a powerful antagonist of both the progesterone and glucocorticoid (GR) receptors. Thus, in some embodiments, the GRA administered to treat an ACTH-secreting tumor is mifepristone, or a salt, tautomer, or derivative thereof. In other embodiments, however, administration of mifepristone is specifically excluded as a GRA for treatment of an ACTH-secreting tumor.

Another 11-β phenyl-aminodimethyl steroid shown to have GR antagonist effects includes the dimethyl aminoethoxyphenyl derivative RU009 (RU39.009), 11-β-(4-dimethyl-aminoethoxyphenyl)-17-α-(propynyl-17-β-hydroxy-4,9-estradien-3-one) (see Bocquel, J. Steroid Biochem. Molec. Biol. 45:205-215, 1993). Another GR antagonist related to RU486 is RU044 (RU43.044) 17-β-hydrox-17-α-19-(4-methyl-phenyl)-androsta-4,9(11)-dien-3-one) (Bocquel, 1993, supra). See also Teutsch, Steroids 38:651-665, 1981; U.S. Pat. Nos. 4,386,085 and 4,912,097.

One embodiment includes compositions that are irreversible anti-glucocorticoids. Such compounds include α-keto-methanesulfonate derivatives of cortisol, including cortisol-21-mesylate (4-pregnene-11-β, 17-α, 21-triol-3, 20-dione-21-methane-sulfonate and dexamethasone-21-mesylate (16-methyl-9-α-fluoro-1,4-pregnadiene-11β, 17-α, 21-triol-3, 20-dione-21-methane-sulfonte). See Simons, J. Steroid Biochem. 24:25-32, 1986; Mercier, J. Steroid Biochem. 25:11-20, 1986; U.S. Pat. No. 4,296,206.

### ii. Modification of the 17-Beta Side Chain Group

Steroidal antiglucocorticoids which can be obtained by various structural modifications of the 17-β side chain are also used in the methods of the invention. This class includes synthetic antiglucocorticoids such as dexamethasone-oxetanone, various 17, 21-acetonide derivatives and 17-beta-carboxamide derivatives of dexamethasone (Lefebvre, 1989, supra; Rousseau, Nature 279:158-160, 1979).

### iii. Other Steroid Backbone Modifications

GRAs used in the various embodiments of the invention include any steroid backbone modification which effects a biological response resulting from a GR-agonist interaction. Steroid backbone antagonists can be any natural or synthetic variation of cortisol, such as adrenal steroids missing the C-19 methyl group, such as 19-nordeoxycorticosterone and 19-norprogesterone (Wynne, Endocrinology 107:1278-1280, 1980).

In general, the 11-βside chain substituent, and particularly the size of that substituent, can play a key role in determining the extent of a steroid's antiglucocorticoid activity. Substitutions in the A ring of the steroid backbone can also be important. For example, 17-hydroxypropenyl side chains can, in some cases, decrease antiglucocorticoid activity in comparison to 17-propynyl side chain containing compounds.

Additional glucocorticoid receptor antagonists known in the art and suitable for practice of the invention include 21-hydroxy-6,19-oxidoprogesterone (See Vicent, Mol. Pharm. 52:749-753, 1997), Org31710 (See Mizutani, J Steroid Biochem Mol Biol 42(7):695-704, 1992), RU43044, RU40555 (See Kim, J Steroid Biochem Mol Biol. 67(3):213-22, 1998), and RU28362.

### b) Non-Steroidal Anti-Glucocorticoids as Antagonists

Non-steroidal glucocorticoid antagonists (GRAs) are also used in the methods of the invention to treat ACTH-secreting tumors in a subject. These include synthetic mimetics and analogs of proteins, including partially peptidic, pseudopeptidic and non-peptidic molecular entities. For example, oligomeric peptidomimetics useful in the invention include (α-β-unsaturated) peptidosulfonamides, N-substituted glycine derivatives, oligo carbamates, oligo urea peptidomimetics, hydrazinopeptides, oligosulfones and the like (See, e.g., Amour, Int. J. Pept. Protein Res. 43:297-304, 1994; de Bont, Bioorganic & Medicinal Chem. 4:667-672, 1996).

Examples of non-steroidal GR antagonists include the GR antagonist compounds disclosed in U.S. Pat. Nos. 5,696,127; 6,570,020; and 6,051,573; the GR antagonist compounds disclosed in US Patent Application 20020077356, the glucocorticoid receptor antagonists disclosed in Bradley et al., J. Med. Chem. 45, 2417-2424 (2002), e.g., 4α(S)-benzyl-2(R)-chloroethynyl-1,2,3,4,4α,9,10,10α(R)-octahydro-phenanthrene-2,7-diol (“CP 394531”) and 4a(S)-benzyl-2(R)-prop-1-ynyl-1,2,3,4,4α,9,10,10α(R)-octahydro-phenanthrene-2,7-diol (“CP 409069”); and the compounds disclosed in PCT International Application No. WO 96/19458, which describes non-steroidal compounds which are high-affinity, highly selective antagonists for steroid receptors, such as 6-substituted-1,2-dihydro-N-protected-quinolines.

In some embodiments, ACTH-secreting tumors are treated with an effective amount of a non-steroidal GRA having a cyclohexyl-pyrimidine backbone, a fused azadecalin backbone, a heteroaryl ketone fused azadecalin backbone, or an octahydro fused azadecalin backbone. For example, the ACTH-secreting tumor can be treated with effective amounts of one of the foregoing GRAs and a somatostatin receptor ligand, somatostatin, or a somatostatin analog. Exemplary GRAs having a cyclohexyl-pyrimidine backbone include those described in U.S. Pat. No. 8,685,973. In some cases, the GRA having a cyclohexyl-pyrimidine backbone has the following structure:

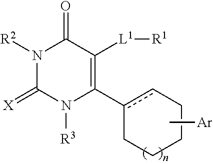

wherein
the dashed line is absent or a bond;
X is selected from the group consisting of 0 and S;
R¹ is selected from the group consisting of cycloalkyl, heterocycloalkyl, aryl and heteroaryl, optionally substituted with from 1 to 3 R^{1a} groups;
each R^{1a} is independently selected from the group consisting of H, C₁₋₆ alkyl, C₂₋₆ alkenyl, C₂₋₆ alkynyl, C₁₋₆ alkoxy, C₁₋₆ alkyl-OR^{1b}, halogen, C₁₋₆ haloalkyl, C₁₋₆ haloaloxy, —OR^{1b}, —NR^{1b}R^{1c}, —C(O)R^{1b}, C(O)OR^{1b}, —OC(O)R^{1b}, —C(O)NR^{1b}R^{1c}, —NR^{1b}C(O)R^{1c}, —SO₂R^{1b}, —SO₂NR^{1b}R^{1c}, cycloalkyl, heterocycloalkyl, aryl and heteroaryl;
R^{1b} and R^{1c} are each independently selected from the group consisting of H and C₁₋₆ alkyl;
R² is selected from the group consisting of H, C₁₋₆ alkyl, C₁₋₆ alkyl-OR^{1b}, C₁₋₆ alkyl-NR^{1b}R^{1c} and C₁₋₆ alkylene-heterocycloalkyl;
R³ is selected from the group consisting of H and C₁₋₆ alkyl;
Ar is aryl, optionally substituted with 1-4 R⁴ groups;
each R⁴ is independently selected from the group consisting of H, C₁₋₆ alkyl, C₁₋₆ alkoxy, halogen, C₁₋₆ haloalkyl and C₁₋₆ haloalkoxy;
L¹ is a bond or C₁₋₆ alkylene; and
subscript n is an integer from 0 to 3,
   or a salts and isomers thereof.

Exemplary GRAs having a fused azadecalin backbone include those described in U.S. Pat. Nos. 7,928,237; and 8,461,172. In some cases, the GRA having a fused azadecalin backbone has the following structure:

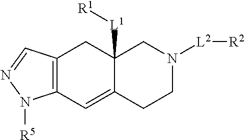

wherein
L¹ and L² are members independently selected from a bond and unsubstituted alkylene;
R¹ is a member selected from unsubstituted alkyl, unsubstituted heteroalkyl, unsubstituted heterocycloalkyl, —OR^{1A}, —NR^{1C}R^{1D}, —C(O)NR^{1C}R^{1D}, and —C(O)OR^{1A}, wherein
R^{1A} is a member selected from hydrogen, unsubstituted alkyl and unsubstituted heteroalkyl,
R^{1C} and R^{1D} are members independently selected from unsubstituted alkyl and unsubstituted heteroalkyl,
wherein R^{1C} and R^{1D} are optionally joined to form an unsubstituted ring with the nitrogen to which they are attached, wherein said ring optionally comprises an additional ring nitrogen;
R² has the formula:

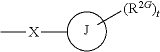

wherein
R^{2G} is a member selected from hydrogen, halogen, unsubstituted alkyl, unsubstituted heteroalkyl, unsubstituted cycloalkyl, unsubstituted heterocycloalkyl, —CN, and —CF₃;
J is phenyl;
t is an integer from 0 to 5;
X is —S(O₂)—; and
R⁵ is phenyl optionally substituted with 1-5 R^{5A} groups, wherein
R^{5A} is a member selected from hydrogen, halogen, —OR^{5A1}, —S(O₂)N^{5A2}R^{5A3}, —CN, and unsubstituted alkyl, wherein
R^{5A1} is a member selected from hydrogen and unsubstituted alkyl, and
R^{5A2} and R^{5A3} are members independently selected from hydrogen and unsubstituted alkyl,
   or salts and isomers thereof.

Exemplary GRAs having a heteroaryl ketone fused azadecalin backbone include those described in U.S. 2014/0038926. In some cases, the GRA having a heteroaryl ketone fused azadecalin backbone has the following structure:

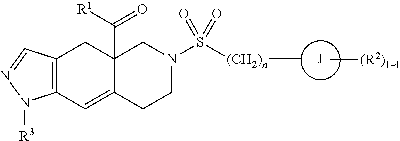

wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, —CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, —NR^{2a}R^{2b}, —C(O)R^{2a}, —C(O)OR^{2a}, —C(O)NR^{2a}R^{2b}, —SR^{2a}, —S(O)R^{2a}, —S(O)₂R^{ea}, C₃₋₈ cycloalkyl, and C₃₋₈ heterocycloalkyl, wherein the heterocycloalkyl groups are optionally substituted with 1-4 R^{2c} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, —CN, and —NR^{2a}R^{2b};
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

Exemplary GRAs having an octohydro fused azadecalin backbone include those described in U.S. Provisional Patent Appl. No. 61/908,333, entitled Octahydro Fused Azadecalin Glucocorticoid Receptor Modulators, Attorney Docket No. 85178-887884 (007800US), filed on Nov. 25, 2013. In some cases, the GRA having an octohydro fused azadecalin backbone has the following structure:

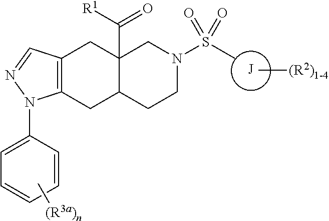

wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl;
ring J is selected from the group consisting of an aryl ring and a heteroaryl ring having from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, —CN, —OH, —NR^{2a}R^{2b}, —C(O)R^{2a}, —C(O)OR^{2a}, —C(O)NR^{2a}R^{2b}, —SR^{2a}, —S(O)R^{2a}, —S(O)₂R^{ea}, C₃₋₈ cycloalkyl, and C₃₋₈ heterocycloalkyl having from 1 to 3 heteroatoms each independently selected from the group consisting of N, O and S;
alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2c} groups;
R^{2a}, R^{2b} and R^{2c} are each independently selected from the group consisting of hydrogen and C₁₋₆ alkyl;
each R^{1a} is independently halogen; and
subscript n is an integer from 0 to 3;
   or salts and isomers thereof.

### C. Somatostatin Receptor Ligands

ACTH-secreting tumors can be treated with an effective amount of a somatostatin receptor ligand such as somatostatin, or a somatostatin analog (SSA). For example, the ACTH-secreting tumor can be treated with effective amounts of a GRA and a somatostatin receptor ligand such as somatostatin, or a somatostatin analog (SSA). In some cases, the somatostatin receptor ligand is a somatostatin receptor agonist.

Exemplary somatostatin receptor ligands include, without limitation, peptide somatostatin receptor ligands, such as those described in U.S. Pat. No. 8,946,154. Exemplary somatostatin receptor ligands further include, without limitation, somatostatin polypeptides from Oncorhynchus mykiss and analogs or derivatives thereof, such as those described in U.S. Pat. No. 6,818,739. Exemplary somatostatin receptor ligands further include, without limitation, antibodies that bind to, or bind to and activate one or more somatostatin receptor subtypes (e.g., any one of SSTR1-5, or a combination thereof). Exemplary somatostatin receptor ligands further include, without limitation, non-peptide somatostatin receptor ligands such as those described in U.S. Pat. No. 7,189,856. Exemplary somatostatin receptor ligands further include, without limitation, the somatostatin receptor ligands described in U.S. Pat. No. 6,358,941.

Exemplary somatostatin receptor ligands further include, without limitation, selective somatostatin receptor ligands. For example, the somatostatin receptor ligand can be selective for (e.g., selectively binds to, or selectively activates) one of SSTR1-5. In some cases, the somatostatin receptor ligand is selective for (e.g., selectively binds to, or selectively activates) SSTR1. In some cases, the somatostatin receptor ligand is selective for SSTR2. Exemplary In some cases, the somatostatin receptor ligand is selective for (e.g., selectively binds to, or selectively activates) SSTR3. In some cases, the somatostatin receptor ligand is selective for (e.g., selectively binds to, or selectively activates) SSTR4. In some cases, the somatostatin receptor ligand is selective for (e.g., selectively binds to, or selectively activates) SSTR5.

In some cases, the somatostatin receptor ligand is selective for (e.g., selectively binds to, or selectively activates) two somatostatin receptors selected from the group consisting of SSTR1-5. For example, the somatostatin receptor ligand can be selective for SSTR1 and 4. As another example, the somatostatin receptor ligand can be selective for SSTR2 and 5. In some cases, the somatostatin receptor ligand is selective for (e.g., selectively binds to, or selectively activates) three somatostatin receptors selected from the group consisting of SSTR1-5. In some cases, the somatostatin receptor ligand is selective for (e.g., selectively binds to, or selectively activates) four somatostatin receptors selected from the group consisting of SSTR1-5. Exemplary selective somatostatin receptor ligands include, without limitation, those described in Rohrer et al., 1998, Science 282:737. Exemplary selective somatostatin receptor ligands further include, without limitation, those described in U.S. Patent Appl. Pub. No. 2006/008299.

In some cases, the somatostatin receptor ligand is selected from the group consisting of octreotide, ¹¹¹In-octreotide, octreotate, pasireotide, lanreotide, and analogs or derivatives thereof. In some cases, the somatostatin receptor ligand is coupled to a detectable label or a cytotoxic agent. Exemplary detectable labels include spin labels, fluorescent labels, and radionuclides. Exemplary cytotoxic agents include radionuclides and cytotoxic chemotherapeutics. Exemplary somatostatin receptor ligands coupled to a radionuclide include, but are not limited to ¹²³I-Tyr³-octreotide, ¹¹¹In-DTPA-D-Phe¹-octreotide, [¹¹¹In-DTPA⁰]octreotide, [⁹⁰Y-DOTA, Tyr³]octreotide, or [¹⁷⁷Lu-DOTA, Tyr³]octreotate.

### D. Methods of Administration

An adrenocorticotropic hormone (ACTH)-secreting tumor can be treated in a subject in need thereof, by simultaneously or sequentially administering to the subject i) a glucocorticoid receptor antagonist (GRA); and ii) somatostatin or a somatostatin analog (SSA), in amounts effective to reduce secretion of ACTH by the tumor. The GRA and somatostatin or SSA can be administered in a single (i.e., combined) dose form, or as a GRA dose and a somatostatin or SSA dose. The GRA can be administered first, followed by a second administration of the somatostatin or SSA. Alternatively, the somatostatin or SSA can be administered first, followed by a second administration of the GRA.

In some cases, the first administration is followed immediately, or nearly immediately, by the second administration. Alternatively, the second administration may be delayed by seconds, minutes, hours, days, or weeks. In some cases, the GRA is repeatedly administered for a period of time (e.g., hours, days, or weeks), and then the somatostatin or SSA is administered (e.g., alone or in combination with a GRA). In some cases, the somatostatin or SSA is repeatedly administered for a period of time (e.g., hours, days, or weeks), and then the GRA is administered (e.g., alone or in combination with a somatostatin or SSA). In some cases, after simultaneous or sequential GRA and somatostatin or SSA administration is performed for a period of time (e.g., hours, days, weeks, or months), the GRA administration is continued for a period of time (e.g., hours, days, weeks, or months) and somatostatin or SSA administration is discontinued for a period of time (e.g., hours, days, weeks, or months).

GRAs can be administered orally. For example, the GRA can be administered as a pill or liquid formulation as described herein. Alternatively, GRAs can be provided via parenteral administration. For example, the GRA can be administered intravenously (e.g., by injection or infusion). Similarly, the somatostatin or SSA can be administered orally, e.g., as a pill or liquid formulation. Alternatively, the somatostatin or SSA can be administered via parenteral administration, e.g., intravenously, subcutaneously, or intramuscularly. Additional methods of administration of the compounds described herein, and pharmaceutical compositions or formulations thereof, are described below in section IV.B.

### E. Methods for Testing Compounds

One or more compounds described herein, or formulations containing one or more compounds described herein, can be tested for glucocorticoid receptor binding or antagonism, somatostatin receptor binding or activation, or a combination thereof. The testing can be performed in vivo or in vitro. In vitro assays can be cell-based assays or biochemical assays (e.g., binding assays) using glucocorticoid receptors or a fragment thereof, somatostatin receptors or a fragment thereof, or combinations thereof.

### 1) Binding Assays

Glucocorticoid receptor antagonists of this invention, both currently known and those later discovered, can be tested for binding activity in a variety of assays. For example, by screening for the ability to compete with a glucocorticoid receptor ligand, such as dexamethasone, for binding to the glucocorticoid receptor. Similarly, somatostatin receptor ligands of this invention, both currently known and those later discovered, can be tested for binding activity, e.g., by screening for the ability to compete with a somatostatin receptor ligand, such as somatostatin, for binding to a somatostatin receptor (e.g., any one of SSTR1-5, or a combination thereof). Those of skill in the art will recognize that there are a number of ways to perform such competitive binding assays.

In some embodiments, glucocorticoid receptor is pre-incubated with a labeled glucocorticoid receptor ligand and then contacted with a test compound. Similarly, a somatostatin receptor can be pre-incubated with a labeled somatostatin receptor ligand and then contacted with a test compound. This type of competitive binding assay may also be referred to herein as a binding displacement assay. Alteration (e.g., a decrease) of the quantity of labeled ligand bound to the receptor indicates that the test compound is a potential receptor agonist or antagonist. In some cases, the labeled ligand is a fluorescently labeled compound. For example, the ligand can be a fluorescently labeled steroid or steroid analog to test for potential glucocorticoid receptor antagonists. Similarly, the ligand can be a fluorescently labeled somatostatin or somatostatin analog to test for potential somatostatin receptor ligands or agonists. Alternatively, the binding of a test compound to the receptor can be measured directly with a labeled test compound. This latter type of assay is called a direct binding assay.

Both direct binding assays and competitive binding assays can be used in a variety of different formats. The formats may be similar to those used in immunoassays and receptor binding assays. For a description of different formats for binding assays, including competitive binding assays and direct binding assays, see Basic and Clinical Immunology 7th Edition (D. Stites and A. Terr ed.) 1991; Enzyme Immunoassay, E. T. Maggio, ed., CRC Press, Boca Raton, Fla. (1980); and “Practice and Theory of Enzyme Immunoassays,” P. Tijssen, Laboratory Techniques in Biochemistry and Molecular Biology, Elsevier Science Publishers B. V. Amsterdam (1985), each of which is incorporated herein by reference.

In solid phase competitive binding assays, for example, the sample compound can compete with a labeled analyte for specific binding sites on a binding agent bound to a solid surface. In this type of format, the labeled analyte can be a glucocorticoid receptor ligand and the binding agent can be glucocorticoid receptor bound to a solid phase. Alternatively, the labeled analyte can be labeled glucocorticoid receptor and the binding agent can be a solid phase glucocorticoid receptor ligand. Similarly, the labeled analyte can be a somatostatin receptor ligand and the binding agent can be a somatostatin receptor bound to a solid phase. Alternatively, the labeled analyte can be labeled somatostatin receptor and the binding agent can be a solid phase somatostatin receptor ligand. The concentration of labeled analyte bound to the capture agent is inversely proportional to the ability of a test compound to compete in the binding assay.

Alternatively, the competitive binding assay may be conducted in liquid phase, and any of a variety of techniques known in the art may be used to separate the bound labeled protein from the unbound labeled protein. For example, several procedures have been developed for distinguishing between bound ligand and excess bound ligand or between bound test compound and the excess unbound test compound. These include identification of the bound complex by sedimentation in sucrose gradients, gel electrophoresis, or gel isoelectric focusing; precipitation of the receptor-ligand complex with protamine sulfate or adsorption on hydroxylapatite; and the removal of unbound compounds or ligands by adsorption on dextran-coated charcoal (DCC) or binding to immobilized antibody. Following separation, the amount of bound ligand or test compound is determined.

Alternatively, a homogenous binding assay may be performed in which a separation step is not needed. For example, a label on the glucocorticoid receptor, or a ligand thereof, may be altered by the binding of the glucocorticoid receptor to its ligand or test compound. Alternatively, a label on a somatostatin receptor, or a ligand thereof, can be altered by the binding of the somatostatin receptor to its ligand or test compound. This alteration in the labeled component can result in a decrease, increase, or change in the signal emitted by label, so that measurement of the label at the end of the binding assay allows for detection or quantitation of the receptor in the bound state. In some cases, a test compound is contacted with a GR in the presence of a fluorescently labeled ligand (e.g., a steroid or steroid analog) with a known affinity for the GR, and the quantity of bound and free labeled ligand is estimated by measuring the fluorescence polarization of the labeled ligand. In some cases, a test compound is contacted with a somatostatin receptor in the presence of a fluorescently labeled somatostatin receptor ligand (e.g., somatostatin or a somatostatin analog) with a known affinity for the somatostatin receptor, and the quantity of bound and free labled ligand is estimated by measuring the fluorescence polarization of the labeled ligand.

A wide variety of labels may be used. The component may be labeled by any one of several methods. Useful radioactive labels include those incorporating ³H, ¹²⁵I, ³⁵S, ¹⁴C, or ³²P. Useful non-radioactive labels include those incorporating fluorophores, chemiluminescent agents, phosphorescent agents, electrochemiluminescent agents, and the like. Fluorescent agents are especially useful in analytical techniques that are used to detect shifts in protein structure such as fluorescence anisotropy and/or fluorescence polarization. The choice of label depends on sensitivity required, ease of conjugation with the compound, stability requirements, and available instrumentation. For a review of various labeling or signal producing systems which may be used, see U.S. Pat. No. 4,391,904, which is incorporated herein by reference in its entirety for all purposes. The label may be coupled directly or indirectly to the desired component of the assay according to methods well known in the art.

High-throughput screening methods may be used to assay a large number of potential modulator compounds. Such “compound libraries” are then screened in one or more assays, as described herein, to identify those library members (particular chemical species or subclasses) that display a desired characteristic activity. Preparation and screening of chemical libraries is well known to those of skill in the art. Devices for the preparation of chemical libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

### 2) Cell-Based Assays

Cell-based assays can involve whole cells or cell fractions containing glucocorticoid receptor to assay for binding or modulation of activity of glucocorticoid receptor by a compound or formulation of the present invention. Alternatively, the cell-based assays can involve whole cells or cell fractions containing a somatostatin receptor (e.g., one or more of SSTR1-5, or a combination thereof) to assay for binding or modulation of activity of somatostatin receptor by a compound or formulation of the present invention. As yet another alternative, cell-based assays can involve whole cells or cell fractions containing both a glucocorticoid receptor and a somatostatin receptor (e.g., one or more of SSTR1-5, or a combination thereof) to assay for binding or modulation of activity of the glucocorticoid receptor and/or somatostatin receptor by a compound or formula of the present invention.

Exemplary cell types that can be used according to the methods of the invention include, e.g., any mammalian cells including leukocytes such as neutrophils, monocytes, macrophages, eosinophils, basophils, mast cells, and lymphocytes, such as T cells and B cells, leukemias, Burkitt's lymphomas, tumor cells (including mouse mammary tumor virus cells), endothelial cells, fibroblasts, cardiac cells, muscle cells, breast tumor cells, ovarian cancer carcinomas, cervical carcinomas, adenocarcimonas, adenomas, pituitary cells, pituitary adenoma or adenocarcinoma cells, neuroendocrine tumor cells, glioblastomas, liver cells, kidney cells, and neuronal cells, as well as fungal cells, including yeast. Cells can be primary cells or tumor cells or other types of immortal cell lines. In some cases, glucocorticoid receptor, somatostatin receptor (e.g., one or more of SSTR1-5, or a combination thereof), or a combination thereof can be expressed in cells that do not express an endogenous version of the receptor(s).

In some cases, fragments of glucocorticoid receptor or somatostatin receptor, as well as protein fusions, can be used for screening. When molecules that compete for binding with receptor ligands are desired, the receptor fragments used can be fragments capable of binding the ligands (e.g., dexamethasone or somatostatin). Alternatively, any receptor fragment of can be used as a target to identify molecules that bind the receptor. Glucocorticoid receptor fragments can include any fragment of, e.g., at least 20, 30, 40, 50 amino acids up to a protein containing all but one amino acid of glucocorticoid receptor. Somatostatin receptor fragments can include any fragment of, e.g., at least 20, 30, 40, 50 amino acids up to a protein containing all but one amino acid of somatostatin receptor.

In some embodiments, a reduction in signaling triggered by glucocorticoid receptor activation is used to identify glucocorticoid receptor antagonists. Signaling activity of glucocorticoid receptor can be determined in many ways. For example, downstream molecular events can be monitored to determine signaling activity. Downstream events include those activities or manifestations that occur as a result of stimulation of a glucocorticoid receptor. Exemplary downstream events useful in the functional evaluation of transcriptional activation and antagonism in unaltered cells include upregulation of a number of glucocorticoid response element (GRE)-dependent genes (PEPCK, tyrosine amino transferase, aromatase). In addition, specific cell types susceptible to GR activation may be used, such as osteocalcin expression in osteoblasts which is downregulated by glucocorticoids; primary hepatocytes which exhibit glucocorticoid mediated upregulation of PEPCK and glucose-6-phosphate (G-6-Pase)). GRE-mediated gene expression has also been demonstrated in transfected cell lines using well-known GRE-regulated sequences (e.g., the mouse mammary tumor virus promoter (MMTV) transfected upstream of a reporter gene construct). Examples of useful reporter gene constructs include luciferase (luc), alkaline phosphatase (ALP) and chloramphenicol acetyl transferase (CAT). The functional evaluation of transcriptional repression can be carried out in cell lines such as monocytes or human skin fibroblasts. Useful functional assays include those that measure IL-lbeta stimulated IL-6 expression; the downregulation of collagenase, cyclooxygenase-2 and various chemokines (MCP-1, RANTES); LPS stimulated cytokine release, e.g., TNFα; or expression of genes regulated by NFkB or AP-1 transcription factors in transfected cell-lines.

In some embodiments, an increase in signaling triggered by somatostatin receptor activation is used to identify somatostatin receptor ligands. Signaling activity of somatostatin receptor can be determined in many ways. For example, downstream molecular events can be monitored to determine signaling activity. Downstream events include those activities or manifestations that occur as a result of stimulation of a somatostatin receptor. Exemplary downstream events useful in the functional evaluation of potential somatostatin receptor antagonists include reduced adenylyl cyclase activity, reduced cyclic AMP or calcium, incrased cGMP, hyperpolarization of potassium (K^{c}) channels, closing of voltage-dependent Ca^{2c} channels, a decrease of intracellular Ca^{2c} influx or concentration, or a combination thereof. Exemplary downstream events useful in the functional evaluation of potential somatostatin receptor antagonists include can additionally or alternatively include activation of SHP1, SHP2, PtPh, Src, Ras, Rasl-GTP, Akt, ERK1/2, p38, ATF2, one or more caspases, p53/Bax, TSC2/TSC1, apoptosis, nitric oxide production, guanylate cyclase, ZAC1 expression, cell cycle arrest, or a combination thereof. Exemplary downstream events useful in the functional evaluation of potential somatostatin receptor antagonists can additionally or alternatively include inhibition of GSK3b, mTOR, cell growth, cell proliferation, hormone secretion, or a combination thereof. Additional signaling pathways of somatostatin receptor activation, including pathways specific to one or more subtypes of somatostatin receptor, are described in Cuevas-Ramos & Fleseriu J. Mol. Endocrinol. (2014) 52, R223-R240.

Compounds that are tested in whole-cell assays can also be tested in a cytotoxicity assay. Cytotoxicity assays are used to determine the extent to which a perceived effect is due to non-glucocorticoid receptor binding cellular effects. In an exemplary embodiment, the cytotoxicity assay includes contacting a constitutively active cell with the test compound. Any decrease in cellular activity indicates a cytotoxic effect.

### 3) Assays for Specificity

The compounds of the present invention may be subject to a specificity assay (also referred to herein as a selectivity assay). Typically, specificity assays include testing a compound that binds glucocorticoid receptor in vitro or in a cell-based assay for the degree of binding to non-glucocorticoid receptor control proteins. Similarly, specficitiy assays can include testing a compound that binds a somatostatin receptor in vitro or in a cell-based assay for the degree of binding to a non-somatostatin receptor control protein, or to a different somatostatin receptor subtype. Selectivity assays may be performed in vitro or in cell based systems, as described above. Binding may be tested against any appropriate control protein, including antibodies, receptors, enzymes, and the like. In an exemplary embodiment, the control protein is a cell-surface receptor or nuclear receptor. In another exemplary embodiment, the control protein is a steroid receptor, such as estrogen receptor, progesterone receptor, androgen receptor, or mineralocorticoid receptor.

### IV. Pharmaceutical Compositions

In some embodiments, the present invention provides a pharmaceutical composition including a compound of the present invention and a pharmaceutically acceptable excipient. In some embodiments, the present invention provides a pharmaceutical composition including a glucocorticoid receptor antagonist of the present invention and a pharmaceutically acceptable excipient. In some embodiments, the present invention provides a pharmaceutical composition including a somatostatin receptor ligand of the present invention and a pharmaceutically acceptable excipient. In some embodiments, the present invention provides a pharmaceutical composition including a glucocorticoid receptor antagonist and a somatostatin receptor ligand of the present invention and a pharmaceutically acceptable excipient.

### A. Formulation

The compositions of the present invention can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and a compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain the compounds of the present invention mixed with a filler or binders such as lactose or starches, lubricants such as talc or magnesium stearate, and, optionally, stabilizers. In soft capsules, the compounds of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution, e.g., in aqueous polyethylene glycol solution.

Aqueous solutions suitable for oral use can be prepared by dissolving one or more compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compounds of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

One or more compositions of the present invention can also be delivered as microspheres for slow release in the body. One or more compositions of the present invention can be delivered as a gel depot for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. In one embodiment, the somatostatin analogue is in the form of a microsphere powder for suspension in a liquid diluent and injection. In some cases, the suspended microsphere formulation provides a long acting release administration form. In one embodiment, the somatostatin analogue is in the form of an autogel, e.g., a supersaturated gel of active ingredient and water. In some cases, the autogel formulation provides a sustained release administration form.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

### B. Administration

One or more compounds or compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds and compositions of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds and compositions of the present invention can be co-administered with other agents. Co-administration includes administering the compound or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the compounds and compositions of the present invention can each be administered once a day, or two, three, or more times per day so as to provide the preferred dosage level per day.

In some embodiments, co-administration can be accomplished by co-formulation, i.e., preparing a single pharmaceutical composition including the compounds and compositions of the present invention and any other agent. Alternatively, the various components can be formulated separately.

The compounds and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, about 50 mg to about 250 mg, or about 75 mg to about 150 mg. Suitable dosages also include about 1, 5, 10, 20, 30, 40, 50, 60, 70, 80, 90, 100, 200, 300, 400, 500, 600, 700, 800, 900 or 1000 mg.

Glucorticoid receptor antagonists (GRAs) can be administered simultaneously or sequentially with a somatostatin receptor ligand (e.g., somatostatin or an analog thereof) at a dose of from about 0.1 mg to about 10,000 mg, about 1 mg to about 1000 mg, about 10 mg to about 750 mg, about 25 mg to about 500 mg, about 50 mg to about 250 mg, or about 75 mg to about 150 mg of the GRA. In some cases, GRAs can be administered simultaneously or sequentially with a somatostatin receptor ligand (e.g., somatostatin or an analog thereof) at a dose of about 1, 5, 10, 20, 30, 40, 50, 60, 70, 80, 90, 100, 200, 300, 400, 500, 600, 700, 800, 900 or 1000 mg of the GRA. In some cases, GRAs can be administered simultaneously or sequentially with a somatostatin receptor ligand (e.g., somatostatin or an analog thereof) at a dose of about 0.1, 0.25, 0.5, 0.75, 1, 1.5, 2, 2.5, 3, 4, 5, 10, 15, 20, 25, 50, 75, 100, 125, or 150 mg/kg of the GRA. In some cases, one or more of the foregoing GRA dosages or a dose within one of the foregoing GRA dose ranges can be administered about four times per day, three times per day, once per day, semi-weekly, weekly, bi-weekly, or monthly. In some cases, a subject is administered a high dose (e.g., 500 mg or more) of GRA for a period of time (e.g., twice per day for one week) and then administered a low dose (e.g., 100 mg or less) of GRA for a period of time.

Somatostatin receptor ligands (e.g., somatostatin or an SSA) can be administered simultaneously or sequentially with a GRA at a dose of from about 0.1 mg to about 10,000 mg, about 1 mg to about 1000 mg, about 10 mg to about 750 mg, about 25 mg to about 500 mg, about 50 mg to about 250 mg, or about 75 mg to about 150 mg of the somatostatin receptor ligand. In some cases, the somatostatin receptor ligand can be administered simultaneously or sequentially with a GRA at a dose of about 1, 5, 10, 20, 30, 40, 50, 60, 70, 80, 90, 100, 200, 300, 400, 500, 600, 700, 800, 900 or 1000 mg of the somatostatin receptor ligand. In some cases, somatostatin receptor ligand can be administered simultaneously or sequentially with a GRA at a dose of about 0.1, 0.25, 0.5, 0.75, 1, 1.5, 2, 2.5, 3, 4, 5, 10, 15, 20, 25, 50, 75, 100, 125, or 150 mg/kg of the somatostatin receptor ligand. In some cases, one or more of the foregoing dosages of somatostatin receptor ligand or a dose within one of the foregoing dose ranges of somatostatin receptor ligand can be administered about four times per day, three times per day, once per day, semi-weekly, weekly, bi-weekly, or monthly. In some cases, a subject is administered a high dose (e.g., 500 mg or more) of somatostatin receptor ligand for a period of time (e.g., twice per day for one week) and then administered a low dose (e.g., 100 mg or less) of somatostatin receptor ligand for a period of time.

The composition can also contain other compatible therapeutic agents. The compounds described herein can be used in combination with one another, with other active agents known to be useful in antagonizing a glucocorticoid receptor, or with adjunctive agents that may not be effective alone, but may contribute to the efficacy of the active agent.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. All publications, patents, and patent applications cited herein are hereby incorporated by reference in their entirety for all purposes.

### V. Examples

### Example 1: Treatment of a Subject with an Ectopic ACTH-Secreting Tumor

A human patient with an ectopic ACTH-secreting pancreatic neuroendocrine tumor metastatic to liver gastrinoma presented with symptoms of ectopic Cushing's Syndrome. The patient was treated with the maximum recommended dose of octreotide long-acting release (LAR), a partial biochemical response was noted (ACTH decreased from 517 pg/mL (113.7 pmol/L) to 345 pg/mL (75.9 pmol/L)), but the Cushing's symptoms were not controlled. After three months of therapy with octreotide LAR, the patient was enrolled in a 24-week, phase 3 clinical trial of mifepristone (MIFE) for inoperable hypercortisolemia.

Prior to the start of MIFE, baseline urinary-free cortisol (UFC) was 2250 mcg/24 hours (6207 nmol/24 hours) and ACTH was 345 pg/mL (75.9 pmol/L). Late-night salivary cortisol (1.71 mcg/dL (47.2 nmol/L)) and serum cortisol (46 mcg/dL (1256 nmol/L)) were also elevated (Table 1). At the time of enrollment, the patient had overtly cushingoid features, including moon facies, plethora, and enlarged dorsocervical and supraclavicular fat pads; purple striae; bruising; edema; and proximal muscle weakness that was so severe that he was unable to rise from a chair without use of his hands. He also had ongoing diabetes, depression, and hypertension associated with hypokalemia.

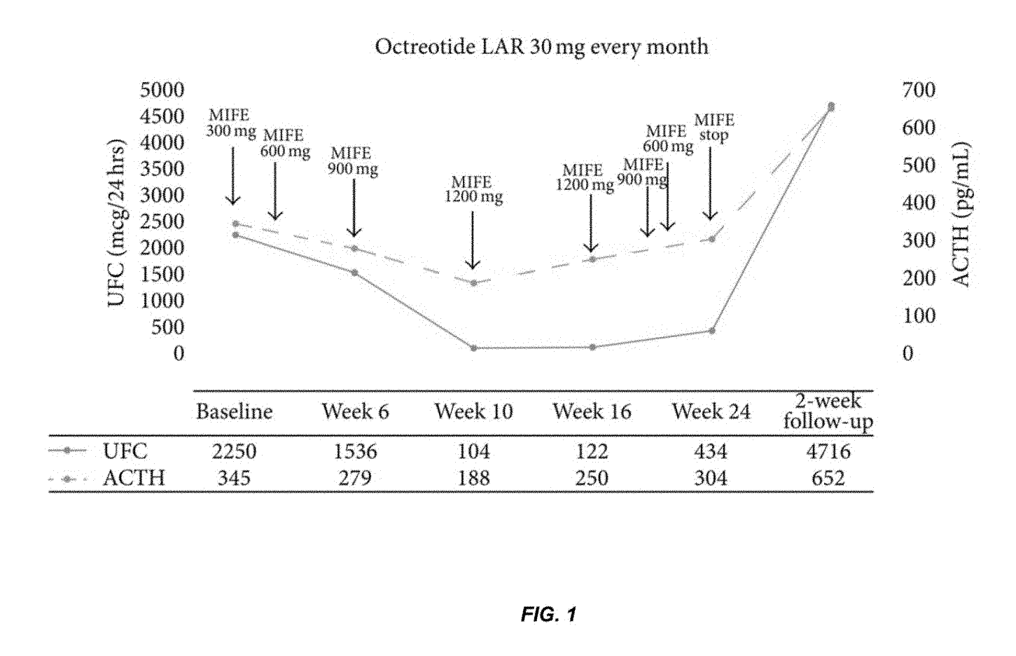

Mifepristone was initiated at a daily dose of 300 mg and gradually increased to 1200 mg per protocol. The patient continued to receive octreotide LAR throughout the duration of the trial. By week 4, insulin therapy was discontinued and by week 12, his cushingoid features essentially resolved. In addition to clinical improvement, a dramatic decrease in cortisol and ACTH was noted during therapy with mifepristone and octreotide LAR (FIG. 1, Table 1). At week 20, mifepristone was briefly stopped for significant fatigue, low appetite, and nausea. Mifepristone was then resumed at a daily dose of 900 mg and 1 week later reduced to 600 mg; no changes were made to octreotide LAR dose. At week 24, his UFC and ACTH levels were 434 mcg/24 hours (1198.7 nmol/24 hours) and 304 pg/mL (66.9 pmol/L), respectively, and mifepristone was stopped per study protocol. During withdrawal of mifepristone, the cortisol and ACTH rose, and 12 days after mifepristone was stopped, clinical signs and symptoms of EAS returned. After 2 weeks, his UFC and ACTH increased to 4716 mcg/24 hours (13016 nmol/24 hours) and 652 pg/mL (143.4 pmol/L), respectively (FIG. 1, Table 1). Mifepristone was resumed for an additional 12-month extension period. Octreotide LAR was discontinued after 2 months and the patient continued with mifepristone for control of his CS-related symptoms. The collection of cortisol and ACTH data was less frequent during the extension study. At the time octreotide was discontinued, the patient's ACTH and serum cortisol were 652 pg/mL (143.4 pmol/L) and 67.8 mcg/dL (1871 nmol/L), respectively. After 12 months in the extension phase, substantial increases in ACTH (3738 pg/mL (822.4 pmol/L)), serum cortisol (135.2 mcg/dL (3732 nmol/L)), and UFC (10716.5 mcg/24 hours (29577.5 nmol/24 hours)) were observed.

TABLE 1
ACTH, UFC, serum cortisol, and salivary cortisol levels
during the course of treatment

| | Baseline (before | | | | | 2-week follow- |
|---|---|---|---|---|---|---|
| Test (normal range) | MIFE) | Week 6 | Week 10 | Week 16 | Week 24 | up (off MIFE) |
| ACTH, pg/mL | 345 | 279 | 188 | 250 | 304 | 652 |
| (7-50 pg/mL) | | | | | | |
| UFC, mcg/24 h | 2250 | 1536 | 104 | 122 | 434 | 4776 |
| (2.0-42.4 mcg/24 h) | | | | | | |
| Serum cortisol, | 46 | 41 | 31 | 31 | 37 | 68 |
| mcg/dL | | | | | | |
| (8 AM, 4.0-22.0 | | | | | | |
| mcg/dL) | | | | | | |
| Late-night salivary | 1.71 | 2.18 | 0.56 | 0.73 | 1.49 | 4.91 |
| cortisol, mcg/dL | | | | | | |
| (10 PM-11 PM, ≤0.09 | | | | | | |
| mcg/dL) | | | | | | |

What is claimed is:

1. A method of controlling hyperglycemia associated with hypercortisolemia in a Cushing's syndrome patient having an adrenocorticotropic hormone (ACTH)-secreting tumor, the method comprising simultaneously or sequentially administering to the subject:
i) a glucocorticoid receptor antagonist (GRA); and
ii) somatostatin or a somatostatin analog (SSA),
thereby controlling said hyperglycemia associated with hypercortisolemia.

2. The method of claim 1, wherein the patient suffers from Cushing's Disease.

3. The method of claim 1, wherein the patient suffers from ectopic ACTH Syndrome.

4. The method of claim 1, wherein the method comprises administering the GRA and SSA for at least two weeks.

5. The method of claim 1, wherein the tumor is a neuroendocrine tumor.

6. The method of claim 1, wherein the glucocorticoid receptor antagonist is a selective inhibitor of the glucocorticoid receptor.

7. The method of claim 1, wherein the glucocorticoid receptor antagonist comprises a steroidal backbone with at least one phenyl-containing moiety in the 11-β position of the steroidal backbone.

8. The method of claim 1, wherein the glucocorticoid receptor antagonist (GRA) is mifepristone.

9. The method of claim 2, wherein the glucocorticoid receptor antagonist (GRA) is mifepristone.

10. The method of claim 1, wherein the glucocorticoid receptor antagonist is selected from the group consisting of 11β-(4-dimethylaminoethoxyphenyl)-17α-propynyl-17β-hydroxy-4,9 estradien-3-one and (17α)-17-hydroxy-19-(4-methylphenyl)androsta-4,9(11)-dien-3-one.

11. The method of claim 2, wherein said glucocorticoid receptor antagonist (GRA) is mifepristone, and the somatostatin or somatostatin analog (SSA) comprises a SSA.

12. The method of claim 1, wherein the glucocorticoid receptor antagonist (GRA) has a non-steroidal backbone.

13. The method of claim 12, wherein the glucocorticoid receptor antagonist (GRA) backbone is a cyclohexyl pyrimidine.

14. The method of claim 13, wherein the cyclohexyl pyrimidine has the following formula: 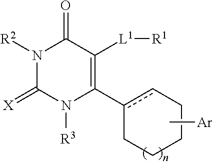
wherein
the dashed line is absent or a bond;
X is selected from the group consisting of O and S;
R¹ is selected from the group consisting of cycloalkyl, heterocycloalkyl, aryl and heteroaryl, optionally substituted with from 1 to 3 R^{1a} groups;
each R^{1a} is independently selected from the group consisting of H, C₁₋₆ alkyl, C₂₋₆ alkenyl, C₂₋₆ alkynyl, C₁₋₆ alkoxy, C₁₋₆ alkyl OR^{1b}, halogen, C₁₋₆ haloalkyl, C₁₋₆ haloaloxy, OR^{1b}, NR^{1b}R^{1c} C(O)R^{1b}, C(O)OR^{1b}, OC(O)R^{1b}, C(O)NR^{1b}R^{1c}, NR^{1b}C(O)R^{1c}, SO₂R^{1b}, SO₂NR^{1b}R^{1c}, cycloalkyl, heterocycloalkyl, aryl and heteroaryl;
R^{1b} and R^{1c} are each independently selected from the group consisting of H and C₁₋₆ alkyl;
R² is selected from the group consisting of H, C₁₋₆ alkyl, C₁₋₆ alkyl-OR^{1b}, C₁₋₆ alkyl NR^{1b}R^{1c} and C₁₋₆ alkylene heterocycloalkyl;
R³ is selected from the group consisting of H and C₁₋₆ alkyl;
Ar is aryl, optionally substituted with 1-4 R⁴ groups;
each R⁴ is independently selected from the group consisting of H, C₁₋₆ alkyl, C₁₋₆ alkoxy, halogen, C₁₋₆ haloalkyl and C₁₋₆ haloalkoxy;
L¹ is a bond or C₁₋₆ alkylene; and
subscript n is an integer from 0 to 3,
or salts and isomers thereof.

15. The method of claim 12, wherein the glucocorticoid receptor antagonist (GRA) backbone is a fused azadecalin.

16. The method of claim 15, wherein the fused azadecalin is a compound having the following formula: 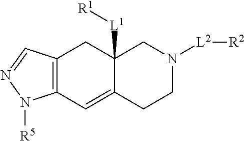
wherein
L¹ and L² are members independently selected from a bond and unsubstituted alkylene;
R¹ is a member selected from unsubstituted alkyl, unsubstituted heteroalkyl, unsubstituted heterocycloalkyl, —OR^{1A}, NR^{1C}R^{1D}, —C(O)NR^{1C}R^{1D}, and —C(O)OR^{1A}, wherein
R^{1A} is a member selected from hydrogen, unsubstituted alkyl and unsubstituted heteroalkyl,
R^{1C} and R^{1D} are members independently selected from unsubstituted alkyl and unsubstituted heteroalkyl,
wherein R^{1C} and R^{1D} are optionally joined to form an unsubstituted ring with the nitrogen to which they are attached, wherein said ring optionally comprises an additional ring nitrogen;
R² has the formula: 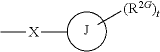
wherein
R^{2G} is a member selected from hydrogen, halogen, unsubstituted alkyl, unsubstituted heteroalkyl, unsubstituted cycloalkyl, unsubstituted heterocycloalkyl, —CN, and —CF₃;
J is phenyl;
t is an integer from 0 to 5;
X is —S(O₂)—; and
R⁵ is phenyl optionally substituted with 1-5 R^{5A} groups, wherein
R^{5A} is a member selected from hydrogen, halogen, —OR^{5A1}, S(O₂)NR^{5A2}R^{5A3}, —CN, and unsubstituted alkyl, wherein
R^{5A1} is a member selected from hydrogen and unsubstituted alkyl, and
R^{5A2} and R^{5A3} are members independently selected from hydrogen and unsubstituted alkyl,
or salts and isomers thereof.

17. The method of claim 12, wherein the glucocorticoid receptor antagonist (GRA) backbone is a heteroaryl ketone fused azadecalin or an octahydro fused azadecalin.

18. The method of claim 17, wherein the heteroaryl ketone fused azadecalin has the formula: 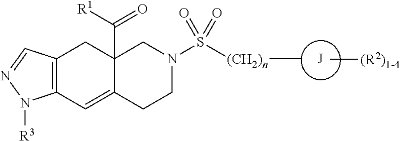
wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, CN, N-oxide, C₃₋₈ cycloalkyl, and C₃₋₈ heterocycloalkyl;
ring J is selected from the group consisting of a cycloalkyl ring, a heterocycloalkyl ring, an aryl ring and a heteroaryl ring, wherein the heterocycloalkyl and heteroaryl rings have from 5 to 6 ring members and from 1 to 4 heteroatoms each independently selected from the group consisting of N, O and S;
each R² is independently selected from the group consisting of hydrogen, C₁₋₆ alkyl, halogen, C_{1 6} haloalkyl, C_{1 6} alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl, wherein the heterocycloalkyl groups are optionally substituted with 1-4 R^{2C} groups;
alternatively, two R² groups linked to the same carbon are combined to form an oxo group (═O);
alternatively, two R² groups are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2d} groups;
R^{2a} and R^{2b} are each independently selected from the group consisting of hydrogen and C₁₋₆ alkyl;
each R^{2c} is independently selected from the group consisting of hydrogen, halogen, hydroxy, C₁₋₆ alkoxy, C₁₋₆ haloalkoxy, CN, and NR^{2a}R^{2b};
each R^{2d} is independently selected from the group consisting of hydrogen and C₁₋₆ alkyl, or two R^{2d} groups attached to the same ring atom are combined to form (═O);
R³ is selected from the group consisting of phenyl and pyridyl, each optionally substituted with 1-4 R^{3a} groups;
each R^{3a} is independently selected from the group consisting of hydrogen, halogen, and C₁₋₆ haloalkyl; and
subscript n is an integer from 0 to 3;
or salts and isomers thereof.

19. The method of claim 17, wherein the octahydro fused azadecalin has the formula: 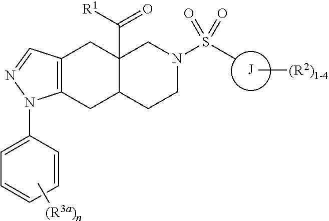
wherein
R¹ is a heteroaryl ring having from 5 to 6 ring members and from 1 to 4 heteroatoms each independently selected from the group consisting of N, O and S, optionally substituted with 1-4 groups each independently selected from R^{1a};
each R^{1a} is independently selected from the group consisting of hydrogen, C₁₋₆ alkyl, halogen, C₁₋₆ haloalkyl, C₁₋₆ alkoxy, C₁₋₆ haloalkoxy, N-oxide, and C₃₋₈ cycloalkyl;
ring J is selected from the group consisting of an aryl ring and a heteroaryl ring having from 5 to 6 ring members and from 1 to 4 heteroatoms each independently selected from the group consisting of N, O and S;
each R² is independently selected from the group consisting of hydrogen, C₁₋₆ alkyl, halogen, C₁₋₆ haloalkyl, C₁₋₆ alkoxy, C₁₋₆ haloalkoxy, C₁₋₆ alkyl-C₁₋₆ alkoxy, CN, OH, NR^{2a}R^{2b}, C(O)R^{2a}, C(O)OR^{2a}, C(O)NR^{2a}R^{2b}, SR^{2a}, S(O)R^{2a}, S(O)₂R^{2a}, C₃₋₈ cycloalkyl, and C₃₋₈ heterocycloalkyl having from 1 to 3 heteroatoms each independently selected from the group consisting of N, O and S;
alternatively, two R² groups on adjacent ring atoms are combined to form a heterocycloalkyl ring having from 5 to 6 ring members and from 1 to 3 heteroatoms each independently selected from the group consisting of N, O and S, wherein the heterocycloalkyl ring is optionally substituted with from 1 to 3 R^{2C} groups;
R^{2a}, R^{2b} and R^{2c} are each independently selected from the group consisting of hydrogen and C₁₋₆ alkyl;
each R^{1a} is independently halogen; and
subscript n is an integer from 0 to 3,
or salts and isomers thereof.

20. The method of claim 1, wherein the somatostatin or somatostatin analog (SSA) comprises a SSA.

21. The method of claim 20, wherein the somatostatin analog (SSA) is selected from the group consisting of octreotide, octreotide, octreotate, pasireotide, lanreotide, and derivatives thereof.

22. The method of claim 20, wherein the somatostatin analog (SSA) is administered in a sustained release formulation.

23. The method of claim 20, wherein the somatostatin analog (SSA) comprises a therapeutic radionuclide.

24. The method of claim 23, wherein the therapeutic radionuclide is selected from the group consisting of ¹¹¹In, ⁹⁰Y, ¹⁷⁷Lu, and ²¹³Bi.

25. The method of claim 1, wherein said ACTH-secretion by said tumor is reduced.

26. The method of claim 2, wherein said ACTH-secretion by said tumor is reduced.

27. The method of claim 1, wherein said ACTH-secreting tumor arises from pituitary corticotroph cells.

28. The method of claim 2, wherein said ACTH-secreting tumor arises from pituitary corticotroph cells.

29. The method of claim 1, wherein said ACTH-secreting tumor is a non-pituitary tumor.

30. The method of claim 2, wherein said ACTH-secreting tumor is a non-pituitary tumor.
